(19) United States
(12) Patent Application Publication
(10) Pub. No.: US 2005/0171413 A1
(43) Pub. Date: Aug. 4, 2005

(54) Integrated device for non-invasive analyte measurement

(75) Inventor: Robert N. Blair, San Jose, CA (US)

Correspondence Address: MORRISON & FOERSTER LLP, 755 PAGE MILL RD, PALO ALTO, CA, 94304-1018, US

(73) Assignee: MedOptix, Inc., Cupertino, CA (US)

(21) Appl. No.: 10/772,529

(22) Filed: Feb. 4, 2004

(51) Int. Cl. A61B 5/00

(52) U.S. Cl. 600/310

(57) ABSTRACT

An integrated device for non-invasive analyte measurement is described herein. In typical operation, the glucose measurement device is self-normalizing in that it does not employ an independent reference sample in its operation. The device uses attenuated total reflection (ATR) infrared spectroscopy for glucose measurement from the user's skin surface. The device also includes a pressure and/or user identification sensor(s) to ensure that an authorized user is utilizing the device. The identification sensor may utilize capacitive or infrared detection of biometric identification features, such as fingerprints, for comparison to a stored value indicative of an authorized user. The device may be configured such that verification of a user's identity may be a prerequisite to use and/or activation of the device.

### FIELD OF THE INVENTION

This invention involves a non-invasive glucose measurement device and a process for determining blood glucose level in the human body using the device. The device also involves detection of pressure and/or verification of the user's identification prior to use of the device.

### BACKGROUND OF THE INVENTION

The American Diabetes Association reports that approximately 6% of the population in the United States, a group of 16 million people, has diabetes, and that it is growing at a rate of 12-15% per annum. The Association further reports that diabetes is the seventh leading cause of death in the United States, contributing to nearly 200,000 deaths per year. Diabetes is a life-threatening disease with broad complications, which include blindness, kidney disease, nerve disease, and heart disease, amputation and stroke. Diabetes is believed to be the leading cause of new cases of blindness in individuals in the range of ages between 20 and 74; from 12,000-24,000 people per year lose their sight because of diabetes. Diabetes is the leading cause of end-stage renal disease, accounting for nearly 40% of new cases. Nearly 60-70% of people with diabetes have mild to severe forms of diabetic nerve damage which, in severe forms, can lead to lower limb amputations. People with diabetes are 2-4 times more likely to have heart disease and to suffer strokes.

Diabetes is a disease in which the body does not produce or properly use insulin, a hormone needed to convert sugar, starches, and the like into energy. Although the cause of diabetes is not completely understood, genetics, environmental factors, and viral causes have been partially identified.

There are two major types of diabetes: Type I and Type II. Type I diabetes (also known as juvenile diabetes) is caused by an autoimmune process destroying the beta cells that secrete the insulin in the pancreas. Type I diabetes most often occurs in young adults and children. People with Type I diabetes must take daily insulin injections to stay alive.

Type II diabetes is a metabolic disorder resulting from the body's inability to make enough, or properly to use, insulin. Type II diabetes accounts for 90-95% of diabetes. In the United States, Type II diabetes is nearing epidemic proportions, principally due to an increased number of older Americans and a greater prevalence of obesity and a sedentary lifestyle.

Insulin, in simple terms, is the hormone that unlocks the cells of the body, allowing glucose to enter those cells and feed them. Since, in diabetics, glucose cannot enter the cells, the glucose builds up in the blood and the body's cells literally starve to death.

Diabetics having Type I diabetes typically are required to self-administer insulin using, e.g., a syringe or a pin with needle and cartridge. Continuous subcutaneous insulin infusion pumps are also available. Insulin itself is typically obtained from pork pancreas or is made chemically identical to human insulin by recombinant DNA technology or by chemical modification of pork insulin. Although there are a variety of different insulins for rapid-, short-, intermediate-, and long-acting forms that may be used variously, separately or mixed in the same syringe, use of insulin for treatment of diabetes is not to be ignored.

It is highly recommended by the medical profession that insulin-using patients practice self-monitoring of blood glucose (SMBG). Based upon the level of glucose in the blood, individuals typically make insulin dosage determinations before injection. Adjustments are necessary since blood glucose levels vary day to day for a variety of reasons, e.g., exercise, stress, rates of food absorption, types of food, hormonal changes (pregnancy, puberty, etc.) and the like. Despite the importance of SMBG, several studies have found that the proportion of individuals who self-monitor at least once a day significantly declines with age. This decrease is likely due simply to the fact that the typical, most widely used, method of SMBG involves obtaining blood from a finger stick. Many patients consider the obtaining of frequent blood samples to be significantly more painful than the self-administration of insulin.

There is a desire for less invasive, and non-invasive, methods of glucose measurement. Methods exist or are being developed for minimally invasive glucose monitoring, which use body fluids other than blood (e.g., sweat, interstitial fluid, or saliva), subcutaneous tissue, or blood measured less invasively.

Subcutaneous glucose measurements seem to lag only a few minutes behind directly measured blood glucose and may actually be a better measurement of the critical values of glucose concentrations in the brain, muscle, and in other tissue. Glucose may be measured by non-invasive or minimally-invasive techniques, such as those making the skin or mucous membranes permeable to glucose or those placing a reporter molecule in the subcutaneous tissue. Needle-type sensors have been improved in accuracy, size, and stability and may be placed in the subcutaneous tissue or peripheral veins to monitor blood glucose with small instruments. See, “An Overview of Minimally Invasive Technologies”, Clin. Chem. 1992 September; 38(9):1596-1600.

Truly simple, non-invasive methods of measuring glucose are not commercially available.

U.S. Pat. No. 4,169,676 to Kaiser, shows a method for the use of ATR glucose measurement by placing the ATR plate directly against the skin and especially against the tongue. The procedure and device shown there uses a laser and determines the content of glucose in a specific living tissue sample by comparing the IR absorption of the measured material against the absorption of IR in a control solution by use of a reference prism. See, column 5, lines 31 et seq.

Swiss Patent No. 612,271, to Dr. Nils Kaiser, appears to be the Swiss patent corresponding to U.S. Pat. No. 4,169,676.

U.S. Pat. No. 4,655,255, to Dähme et al., describes an apparatus for non-invasively measuring the level of glucose in a blood stream or tissues of patients suspected to have diabetes. The method is photometric and uses light in the near-infrared (“NIR”) region. Specifically, the procedure uses light in the 1,000 to 2,500 nm range. Dähne's device is jointly made up to two main sections, a light source and a detector section. They may be situated about a body part such as a finger. The desired near-infrared light is achieved by use of filters. The detector section is made up of a light-collecting integrating sphere or half-sphere leading to a means for detecting wavelengths in the near-infrared region. Dähne et al. goes to some lengths teaching away from the use of light in the infrared range having a wavelength greater than about 2.5 micrometers since those wavelengths are strongly absorbed by water and have very little penetration capability into living tissues containing glucose. That light is said not to be “readily useable to analyze body tissue volumes at depths exceeding a few microns or tens of microns.” Further, Dähne et al. specifically indicates that an ATR method which tries to circumvent the adverse consequences of the heat effect by using a total internal reflection technique is able only to investigate to tissue depths not exceeding about 10 micrometers, a depth which is considered by Dähne et al. to be “insufficient to obtain reliable glucose determination information.”

U.S. Pat. No. 5,028,787, to Rosenthal et al., describes a non-invasive glucose monitoring device using near-infrared light. The light is passed into the body in such a way that it passes through some blood-containing region. The so-transmitted or reflected light is then detected using an optical detector. The near-infrared light sources are preferably infrared emitting diodes (IRED). U.S. Pat. No. 5,086,229 is a continuation in part of U.S. Pat. No. 5,028,787.

U.S. Pat. No. 5,178,142, to Harjunmaa et al, teaches the use of a stabilized near-infrared radiation beam containing two alternating wavelengths in a device to determine a concentration of glucose or other constituents in a human or animal body. Interestingly, one of the transmitted IR signals is zeroed by variously tuning one of the wavelengths, changing the extracellular to intracellular fluid ratio of the tissue by varying the mechanical pressure on a tissue. Or, the ratio may be allowed to change as a result of natural pulsation, e.g., by heart rate. The alternating component of the transmitted beam is measured in the “change to fluid ratio” state. The amplitude of the varying alternating signal is detected and is said to represent glucose concentration or is taken to represent the difference in glucose concentration from a preset reference concentration.

U.S. Pat. No. 5,179,951 and its divisional, U.S. Pat. No. 5,115,133, to Knudson, show the application of infrared light for measuring the level of blood glucose in blood vessels in the tympanic membrane. The detected signal is detected, amplified, decoded, and, using a microprocessor, provided to a display device. The infrared detector (No. 30 in the drawings) is said simply to be a “photo diode and distance signal detector” which preferably includes “means for detecting the temperature of the volume in the ear between the detector and the ear's tympanic membrane.” Little else is said about the constituency of that detector.

U.S. Pat. No. 5,433,197, to Stark, describes a non-invasive glucose sensor. The sensor operates in the following fashion. A near-infrared radiation is passed into the eye through the cornea and the aqueous humor, reflected from the iris or the lens surface, and then passed out through the aqueous humor and cornea. The reflected radiation is collected and detected by a near-infrared sensor which measures the reflected energy in one or more specific wavelength bands. Comparison of the reflected energy with the source energy is said to provide a measure of the spectral absorption by the eye components. In particular, it is said that the level of glucose in the aqueous humor is a function of the level of glucose in the blood. It is said in Stark that the measured glucose concentration in the aqueous humor tracks that of the blood by a fairly short time, e.g., about 10 minutes. The detector used is preferably a photodiode detector of silicon or InGaAs. The infrared source is said preferably to be an LED, with a refraction grating so that the light of a narrow wavelength band, typically 10 to 20 nanometers wide, passes through the exit slit. The light is in the near-infrared range. The use of infrared regions below 1400 nanometers and in the region between 1550 and 1750 nanometers is suggested.

U.S. Pat. No. 5,267,152, to Yang et al., shows a non-invasive method and device for measuring glucose concentration. The method and apparatus uses near-infrared radiation, specifically with a wavelength of 1.3 micrometers to 1.8 micrometers from a semiconductor diode laser. The procedure is said to be that the light is then transmitted down through the skin to the blood vessel where light interacts with various components of the blood and is then diffusively reflected by the blood back through the skin for measurement.

Similarly, U.S. Pat. No. 5,313,941, to Braig et al., suggests a procedure and apparatus for monitoring glucose or ethanol and other blood constituents in a non-invasive fashion. The measurements are made by monitoring absorption of certain constituents in the longer infrared wavelength region. The long wavelength infrared energy is passed through the finger or other vascularized appendage. The infrared light passing through the finger is measured. The infrared source is pulsed to prevent burning or other patient discomfort. The bursts are also synchronized with the heartbeat so that only two pulses of infrared light are sent through the finger per heartbeat. The detected signals are then analyzed for glucose and other blood constituent information.

U.S. Pat. No. 5,398,681, to Kuperschmidt, shows a device which is said to be a pocket-type apparatus for measurement of blood glucose using a polarized-modulated laser beam. The laser light is introduced into a finger or ear lobe and the phase difference between a reference signal and the measurement signal is measured and processed to formulate and calculate a blood glucose concentration which is then displayed.

U.S. Pat. No. 6,001,067 shows an implantable device suitable for glucose monitoring. It utilizes a membrane which is in contact with a thin electrolyte phase, which in turn is covered by an enzyme-containing membrane, e.g., glucose oxidase in a polymer system. Sensors are positioned in such a way that they measure the electrochemical reaction of the glucose within the membranes. That information is then passed to the desired source.

None of the cited prior art suggests the device and method of using this device described and claimed below.

### SUMMARY OF THE INVENTION

In using the glucose or analyte level measurement device described herein, the identification of the user prior to use of the device may be performed to ensure that the device, which is preferably calibrated to a specific user, may not be used inadvertently or otherwise by a person other than the specific user. The glucose measurement device utilizes infrared (IR) attenuated total reflection (ATR) spectroscopy. The device itself preferably comprises an IR source for emitting an IR beam into the ATR plate, the ATR plate against which the sampled human skin surface is pressed, and at least one IR sensor for measuring the absorbance of two specific regions of the IR spectrum, e.g., “referencing wavelengths” and “measuring wavelengths.” The IR source emits IR radiation at least in the region of the referencing wavelength and the measuring wavelength. For glucose, one such referencing wavelength is between about 8.25 micrometers and about 8.75 micrometers and one such measuring wavelength is between about 9.50 micrometers and about 10. 00 micrometers. The IR sources may be broadband IR sources, non-laser sources, or two or more selected wavelength lasers.

The ATR plate is configured to permit multiple internal reflections, perhaps about 3 to about 25 internal reflections or more, against the measurement surface prior to measurement by the IR sensors. Once the reflected beams are measured by the IR sensor(s), the resulting signals may then be transformed using analog comparators and/or digital computers into readable or displayable values. A normalizing step practiced by simultaneously detecting and quantifying the referencing and measuring wavelength components prior to contacting the skin surface is also desirable.

In general, the device may be used in the following manner: a skin surface on a human being, for instance, the skin of the finger, is placed on the ATR plate. The skin surface is radiated with an IR beam having components at least in the two IR regions as described above as “referencing wavelengths” and “measuring wavelengths.” The beam which ultimately is reflected out of the ATR plate then contains information indicative of the blood glucose level in the user. As noted above, it is also desirable to maintain that skin surface on the ATR plate at a relatively constant pressure that is typically above a selected minimum pressure. This may be done manually or by measuring and maintaining the pressure or monitoring the constancy of a selected IR value with the sensor assembly

The device may also utilize a biometric user-identification (ID) methodology, such as fingerprint identification. This user-identification may be implemented as an integrated feature in the device. Similarly, the user-identification may be separate from, or integrated with, with a pressure sensor. As mentioned above, identification of a user prior to use of the device may be performed to ensure that a device, which is preferably calibrated to a specific user, may not be used inadvertently or otherwise by a person other than the specific user.

The sensor assembly for measuring a pressure exerted by the user upon the device and/or the user's biometric information may be integrated within an ATR/sensor assembly. In one variation, the sensor assembly may be positioned adjacent to the ATR crystal. In another variation, the sensor assembly may be integrated along the length of the ATR crystal (e.g., either along one or both sides) in a continuous or discontinuous fashion. In yet another variation, the sensor assembly may be integrated directly within the crystal body. In each of these variations, the upper surface of the ATR crystal and the contact surface of the sensor assembly preferably form a single continuous surface upon which the user may place a skin surface, e.g., a finger. Moreover, the sensor assembly preferably has a width which is wide enough to accommodate and contact at least a sufficient portion of the skin surface for pressure detection and/or at least enough of the skin surface to enable a determination of the user's identification, e.g., having an area of the contacted skin surface with sufficient identifying fingerprint patterns to distinguish one user from another user. However, the sensor assembly may be of a variety of different forms and sizes. For example, the sensor assembly may be of a horseshoe configuration, may be configured as a series of strips, may be circular in nature, or any combinations thereof.

Verification of a user's identification may be tied to the operation of the device such that a measurement will not be taken until the proper identification of the user has been verified by the device. Verification of a user's identification prior to use of the device may be incorporated as an optional safeguard. For instance, identification may be performed to ensure that the device, which is preferably calibrated to a specific user, may not be used inadvertently or otherwise by a person other than the specified user. Thus, the sensor assembly described above may be configured in one variation as a fingerprint sensing device.

One variation for biometric evaluation of a user may include fingerprint detection by utilizing a capacitance map of the tissue in contact with the sensor assembly. Such a capacitance sensor assembly may have a plurality of sensor cells in the form of an array upon a substrate. The array may be organized in a column/row fashion to sense a fingerprint pattern of a finger, or a portion of a finger or some other body part, which may be placed onto the substrate. Each of the sensor cells forming the array may be configured with electronics to detect the presence or absence of a ridge or a valley of a fingerprint pattern placed on top of the substrate. Each sensing cell may provide an output indicative of the portion of the fingerprint pattern detected by the sensor cell and a composite signal from the array of sensor cells may then provide an output indicative of a particular fingerprint pattern. This detected pattern may be compared to a particular user's stored fingerprint pattern.

Aside from capacitive sensing, another variation may utilize visible or infrared light (for instance, mid-infrared light) to illuminate the user's finger to capture a reflected fingerprint as an image. This captured image may be compared to a stored fingerprint image of a specified person to verify the user's identification. The light source utilized may be the same light source used to transmit the light into the ATR crystal or it may be a separate light source dedicated to the sensor assembly. The reflected light may be incident upon a photosensor or light detector, e.g., a CCD or CMOS imaging system, and the incident light may then be transmitted as electronic signals to a processor for image processing and comparison. Optionally, a transmitter may be used to transmit a detected fingerprint image to an external receiving unit where the detected image may be compared to an externally stored image, the results of which may then be transmitted back to the sensor assembly for processing by a processor.

As mentioned above, both user identification and pressure may be detected by appropriate sensors integrated into the glucose measurement device. In one variation, a detection algorithm may be utilized with the device sensors. When a user places a finger onto the ATR crystal and/or sensor assembly, the user's identification may then be detected or sensed. If a positive match is not detected, this may indicate that an improper or inadequate fingerprint measurement has occurred or an unauthorized user has attempted to use the device. Thus, the device may be configured to not operate until a positive match has been detected. If the match is positive, this may indicate that an authorized user having the appropriate stored profile has been detected. The device may then detect whether the user is exerting the adequate amount of pressure onto the ATR crystal. If the user is not exerting the appropriate amount of pressure, the device may be configured to await measurement or activation until the minimum adequate pressure is sensed. Alternatively, the device may be configured to reset and re-verify the user's identification first before detecting the pressure again, as shown. This optional step may be utilized to prevent an authorized user from activating the device with his/her verified identification and then passing the device to an unauthorized user for glucose or analyte measurement. Finally, once the adequate pressure has been detected, the device may operate as described herein to detect and measure the analyte or glucose of the user. Various combinations as well as variations on the order of detection and/or identification may be utilized, depending upon the desired results.

### BRIEF DESCRIPTION OF THE DRAWINGS

FIGS. 1A, 1B, 1C, and 1D show side views of various ATR plates and their general operation.

FIG. 2 shows an IR spectrum of d-glucose.

FIGS. 3A and 3B show schematic side and top views of one variation of an ATR crystal and an integrated pressure and/or ID sensor.

FIG. 4A shows a schematic side view of another variation of an integrated ATR crystal and pressure and/or ID sensor.

FIGS. 4B and 4C show top views of variations of the integrated sensor of FIG. 4A.

FIGS. 5A and 5B show schematic side and top views of another variation of an ATR crystal with an integrated pressure and/or ID sensor.

FIG. 6 shows a schematic layout of the optics suitable for use with the inventive device.

FIG. 7 shows a packaged variation of the inventive glucose measuring device.

FIG. 8 shows a graph of pressure on the ATR crystal vs. IR value.

FIG. 9 shows an illustrative detailed top view of one variation of a sensor assembly array which may be integrated into the ATR crystal.

FIG. 10 shows an illustrative close-up side view of a sensor assembly interacting with a user's finger.

FIG. 11 shows an illustrative side view of a sensor assembly in relation to the unique ridges and valleys defined by the user's fingerprint.

FIG. 12 shows a schematic illustration of a variation on an infrared sensor assembly.

FIG. 13 shows a schematic illustration of another variation on an infrared sensor assembly.

FIG. 14 shows a variation of a detection algorithm which may be utilized with the device.

### DETAIL DESCRIPTION OF THE INVENTION

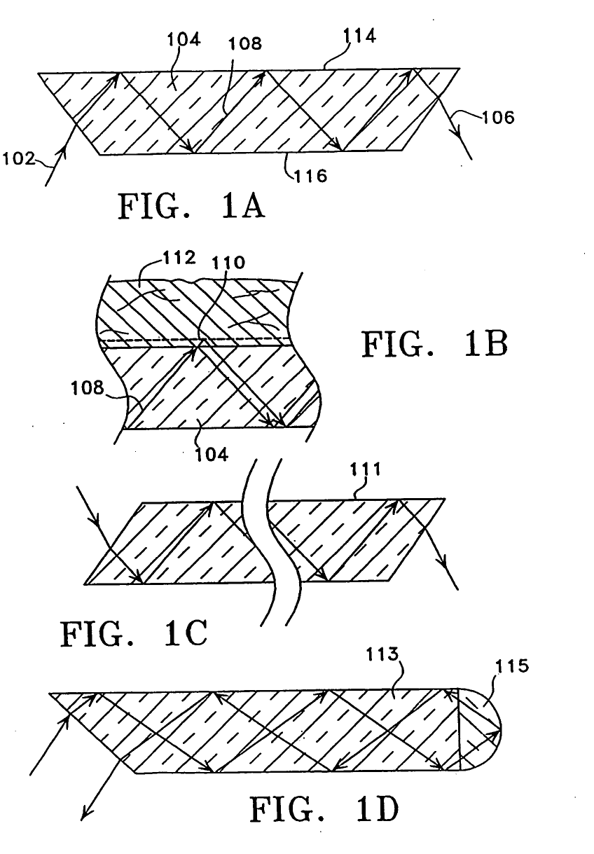

The device in this invention uses infrared (“IR”) attenuated total reflectance (“ATR”) spectroscopy to detect and ultimately to determine the level of a selected analyte, such as, blood glucose, in the human body. Preferably, the inventive device uses an ATR procedure in which the size and configuration of the crystal permits a number of internal reflections before the beam is allowed to exit the crystal with its measured information. In general, as shown in FIGS. 1A and 1B, when an infrared beam 102 is incident on the upper surface of the ATR crystal 104—or ATR plate—at an angle which exceeds a critical angle Θ_{C}, the beam 102 will be totally internally reflected within crystal 104. Each reflection of the beam within the ATR plate, and specifically against the upper surface 114, provides an incremental increase in the information about the composition of the sample 112 resting against that upper surface 114. The more numerous the reflections, the more likely accurate readings are obtained. The incident beam 102 becomes reflected beam 106 as it exits crystal 104 as shown in FIG. 1A. Higher refractive index materials are typically chosen for the ATR crystal to minimize the critical angle. The critical angle is a function of the refractive indices of both the sample and the ATR crystal and is defined as:

$${Θ}_{C} = {sin}^{- 1} ( \frac{{n}_{2}}{{n}_{1}} )$$

Here, n₁ is the refractive index of the ATR crystal and n₂ is the refractive index of the sample.

Throughout this specification, we refer to wavelength measures as specific values. It should be understood that we intend those values to be bands or ranges of values, typically with a tolerance of ±0.20 micron, preferably ±0.10 micron. For instance, a value of 8.25 microns would mean a band of 8.15 to 8.35 microns, and perhaps 8.05 to 8.45 microns depending upon the context.

As shown in FIG. 1B, the internally reflected beam 108 includes an evanescent wave 110 which penetrates a short distance into sample 112 over a wide wavelength range. In those regions of the IR spectrum in which the sample absorbs IR, some portion of the light does not reach the sensor. It is these regions of IR absorbance which provide information, in this inventive device, for quantification of the glucose level.

We have found that the mid-IR spectrum does not penetrate into the skin to an appreciable level. Specifically, the skin is made up of a number of layers: the outermost—the stratum corneum—is a layer substantially free of cholesterol, water, gamma globulin, albumin, and blood. It is a shallow outer region covering the stratum granulosum, the stratum spinosum, and the basal layer. The area between the basal layer to the outside is not vascularized. It is unlikely that any layer other than the stratum corneum is traversed by the mid-IR light involved in this inventive device. Although we do not wish to be bound by theory, it is likely that the eccrine or sweat glands transport at least some of the glucose to the outer skin layers for measurement and analysis by our inventions.

We prefer the use of higher refractive index crystals such as zinc selenide, zinc sulfide, diamond, germanium, and silicon as the ATR plate. The index of refraction of the ATR plate (104) should be significantly higher than that of the sample 112. In some variations, the ATR plate is made from zinc selenide, in other variations the ATR plate is made from diamond (natural and synthetic versions). In other variations the ATR plate is made from germanium.

Further, the ATR crystal 104 shown in FIG. 1A is shown to be trapezoidal and having an upper surface 114 for contact with the sample, which sample, in this case, is skin from a living human body. However, this shape is only for the purposes of mechanical convenience and ease of application into a working commercial device. Other shapes, in particular, a parallelogram 111 such as shown in FIG. 1C and the reflective crystal 113 shown in FIG. 1D having mirrored end 115, are also quite suitable for this inventive device should the designer so require. The mirrored reflective crystal 113 has both an IR source and the IR sensors at the same end of the crystal.

It is generally essential that the ATR crystal or plate 104 have a sample or upper surface 114 which is essentially parallel to the lower surface 116. In general, the ATR plate 104 is preferably configured and utilized so that the product of the practical number of internal reflections of internal reflected beam 108 and the skin penetration per reflection of this product is optimized. When optimizing this product, called the effective pathlength (EPL), the information level in beam 106 as it leaves ATR plate 104 is significantly higher. Further, the higher the value of the index of refraction, n₂, of the ATR plate 104, the higher is the number of internal reflections. The sensitivity of the IR sensors also need not be as high when the EPL is optimized. We consider the number of total reflections within the crystal to be preferably from about 3 to about 25 or more for adequate results.

We have surprisingly found that a glucose measuring device made according to this invention is quite effective on the human skin of the hands and fingers. We have found that the glucose concentration as measured by the inventive devices correlates very closely with the glucose concentration determined by a direct determination from a blood sample. As will be discussed below, the glucose level as measured by the inventive device also is surprisingly found closely to track the glucose level of blood in time as well. This is surprising in that the IR beam likely passes into the skin, i.e., the stratum corneum, for only a few microns. It is unlikely in a fingertip that any blood is crossed by that light path. As discussed above, the stratum corneum is the outer layer of skin and is substantially unvascularized. The stratum corneum is the final outer product of epidermal differentiation or keratinization. It is made up of a number of closely packed layers of flattened polyhedral corneocytes (also known as squames). These cells overlap and interlock with neighboring cells by ridges and grooves. In the thin skin of the human body, this layer may be only a few cells deep, but in thicker skin, such as may be found on the toes and feet, it may be more than 50 cells deep. The plasma membrane of the corneocyte appears thickened compared with that of keratinocytes in the lower layers of the skin, but this apparent deposition of a dense marginal band formed by stabilization of a soluble precursor, involucrin, just below the stratum corneum.

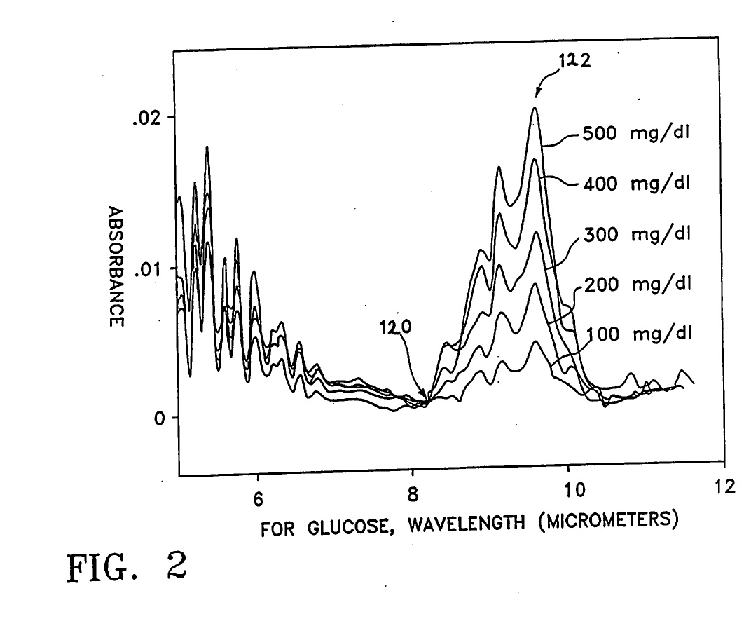

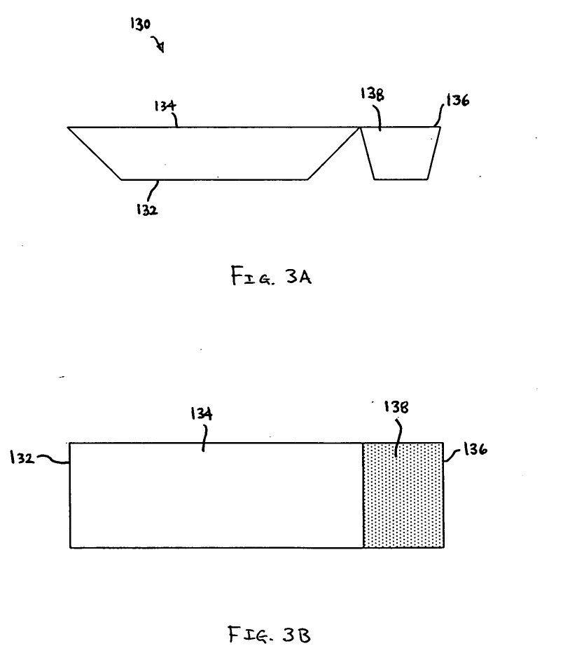

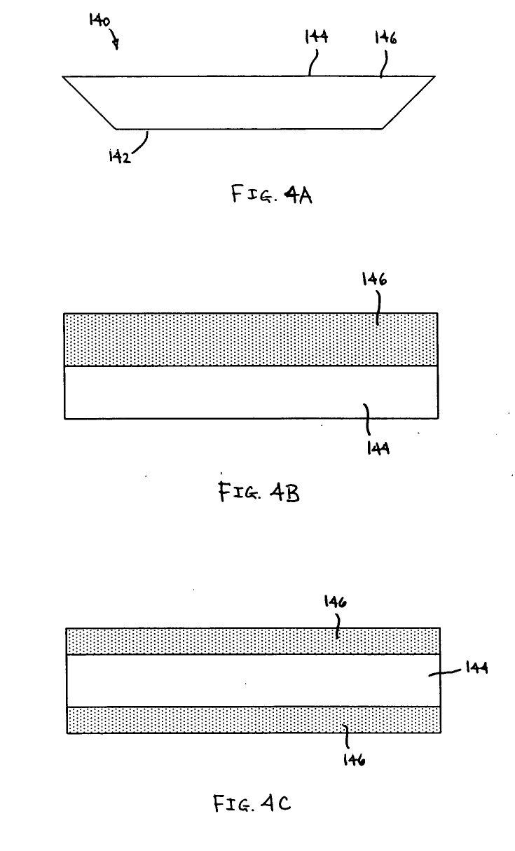

Additionally, the inventive device can be highly simplified compared to other known devices in that the device can be “self-normalizing” due to the specifics of the IR signature of glucose. FIG. 2 shows the IR absorbance spectra of d-glucose. The family of curves there depicted demonstrates that in certain regions of the IR spectrum, there is a correlation between absorbance and the concentration of glucose. Further, there is a region in which the absorbance is not at all dependent upon the concentration of glucose. Our device, in its preferable method of use, uses these two regions of the IR spectra. These regions are in the so-called mid-IR range, i.e., wavelengths between approximately 2.5 and 14 micrometers. In particular, the “referencing wavelength” point is just above 8 micrometers 120, e.g., 8.25 to 8.75 micrometers, and the pronounced peaks 122 at the region between about 9.50 and 10.00 micrometers is used as a “measuring wavelength”. The family of peaks 122 may be used to determine the desired glucose concentration.

Use of the two noted IR regions is also particularly suitable since other components typically found in the skin, e.g., water, cholesterol, etc., do not cause significant measurement error when using the method described herein.

We have found that it is desirable to maintain a minimum threshold pressure on the body part, e.g., the finger, which is to be used as the area for measurement. Generally, a variance in the pressure does not shift the position of the detected IR spectra, but it may affect the sensitivity of the overall device. One variation of the device may utilize an ATR crystal configured with an integrated pressure sensor to measure the pressure exerted by a user upon the device. The pressure sensor may be electrically connected to the device and optionally configured such that a measurement will not be taken until a desired minimum pressure exerted by the user has been measured by the pressure sensor.

Another variation of the device may utilize a biometric user-identification methodology, such as fingerprint identification. The user-identification may be implemented as an integrated feature in the device separate from or in combination with a pressure sensor. The verification of a user's identification may be an optional integrated feature configured such that a measurement will not be taken by the device until the proper identification of the user has been verified by the device, as described in further detail below. Identification of a user prior to use of the device may be performed to ensure that a device, which is preferably calibrated to a specific user, may not be used inadvertently or otherwise by a person other than the specific user.

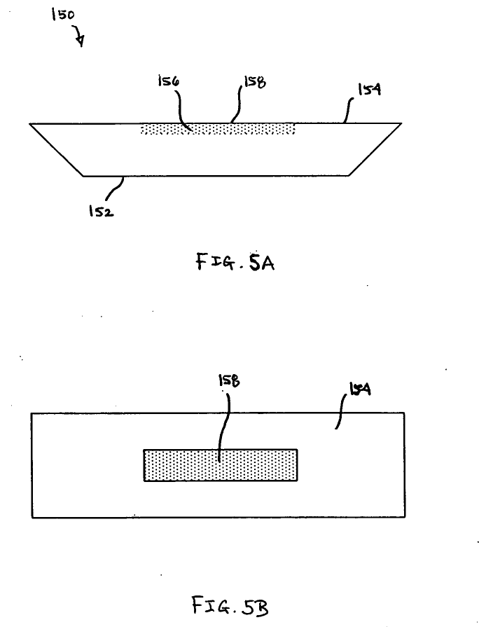

FIGS. 3A and 3B show schematic side and top views of one variation of how an ATR crystal and pressure and/or ID sensor may be integrated. FIG. 3A shows ATR/sensor assembly 130 where sensor assembly 136 may be positioned adjacently to ATR crystal 132. Sensor assembly 136 may define a contact surface 138 which is preferably flush with upper surface 134 defined by ATR crystal 132 such that a single continuous surface may be defined upon which the user may place, e.g., a finger. FIG. 3B is a top view of ATR/sensor assembly 130 showing one example of how contact surface 138 may be aligned with upper surface 134. Examples of devices and methods of use for sensor assembly 136 is described in greater detail below.

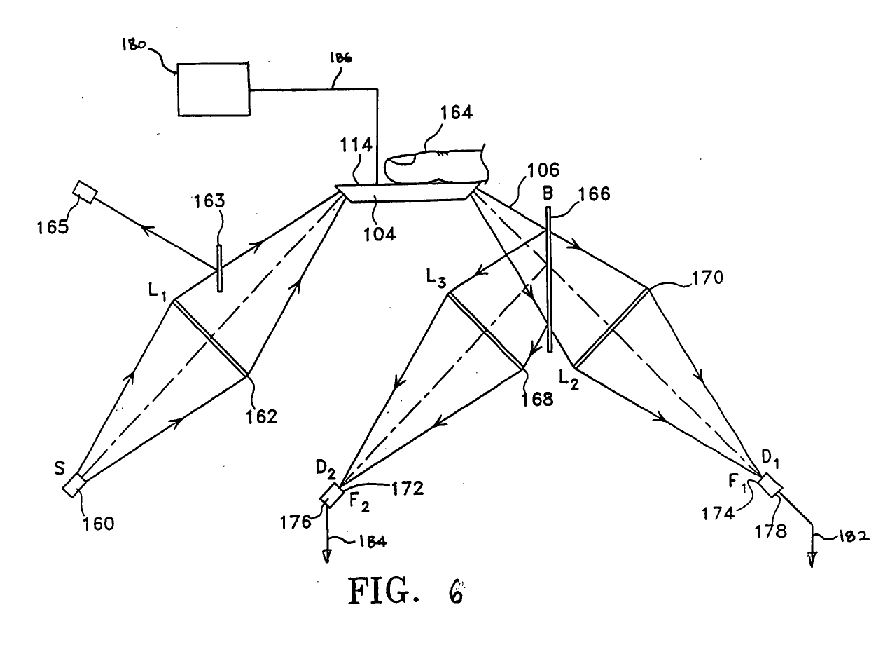

FIG. 4A shows a side view of another variation 140 of an ATR crystal 142 having an integrated pressure or ID sensor. FIGS. 4B and 4C show top views of alternative variations of pressure and/or ID sensors which may be integrated along the length of the ATR crystal 142. FIG. 4B, for instance, shows a variation in which a contact surface 146 of the pressure and/or ID sensor may be adjacent along the length of a single side of the ATR crystal upper surface 144. FIG. 4C shows another variation similar to that of FIG. 4B; however, in this variation the contact surfaces 146 may be located along the length of both sides of the ATR crystal upper surface 144. In either variation, the contact surfaces 146 preferably has a width which is wide enough to accommodate and contact at least a sufficient portion of the skin surface for pressure detection and/or at least enough of the skin surface to enable a determination of the user's identification, e.g., having an area of the contacted skin surface with sufficient identifying fingerprint patterns to distinguish one user from another user.

FIGS. 5A and 5B show side and top views of yet another crystal/sensor variation 150. ATR crystal 152 in this variation may have sensor assembly 156 integrated directly within the crystal 152 such that the contact surface 158 of sensor assembly 156 is flush with upper surface 154 of ATR crystal 152. Moreover, in this variation, sensor assembly 156 may be completely surrounded by ATR crystal 152 except for the exposed contact surface 158.

FIG. 6 shows an optical schematic of a variation of the assembly. ATR crystal 104 with sample side 114 is shown and IR source 160 is provided. ATR crystal 104 may have a pressure and/or identification sensor integrated in any number of configurations as described above or as known by one of skill in the art. IR source 160 may be any of a variety of different kinds of sources, for instance, a broadband IR source, one having radiant temperatures of 300° C. to 800° C., or a pair of IR lasers selected for the two regions of measurement discussed above, or other suitably emitted or filtered IR light sources. Lens 162, for focusing light from IR source 160 into ATR plate 104, is also shown. An optional mirror 163 may be included to intercept a portion of the beam before it enters the ATR plate 104 and then to measure the strength or intensity of that beam in IR sensor 165. Measurement of that incident light strength or intensity (during normalization and during the sample measurement) assures that any changes in that value can be compensated for.

The light then passes into ATR plate 104 for contact with a body part 164, shown in this instance to be the desired finger. The reflected beam 106 exits ATR plate 104 and may then be split using beam splitter 166. Beam splitter 166 simply transmits some portion of the light through the splitter and reflects the remainder. The two beams may then be passed through, respectively, lenses 168 and 170. The so-focused beams are then passed to a pair of sensors which are specifically selected for detecting and measuring the magnitude of the two beams in the selected IR regions. Generally, the sensors will be made up of filters 172 and 174 with light sensors 176 and 178 behind. Generally, one of the filters 172, 174 will be in the region of the referencing wavelength and the other will be in that of the measuring wavelength.

A more detailed description of the general operation of the ATR assembly may be seen in U.S. Pat. No. 6,424,851 (Berman et al.) entitled “Infrared ATR Glucose Measurement System (II)”. Also, additional methods or devices may be employed to improve the detected signals; for instance, lock-in amplifiers may be in electrical communication with the sensors 176 and 178 along with a modulator for modulating the light source 160. The use of lock-in amplifiers is described in further detail in U.S. patent application Ser. No. 10/434,963 entitled “Non-Invasive Analyte Measurement Device Having Increased Signal To Noise Ratios” filed May 9, 2003. Furthermore, although two separate sensors 116, 178 are shown in the figure, other variations may include the use of a single sensor or detector, as described in further detail in U.S. patent application Ser. No. 10/739,657 entitled “Single Detector Infrared ATR Glucose Measurement System”, filed Dec. 17, 2003. Each of these references described above is co-owned and incorporated herein by reference in its entirety.

The integrated pressure and/or identification sensor within ATR plate 104 may be electrically connected via electrical line 186 to a processor 180 for measurement and/or calculation of the detected pressure and/or identification parameters. Light sensors 176 and 178 may also be electrically connected via electrical lines 184 and 182, respectively, to corresponding lock-in amplifiers and/or directly to processor 180.

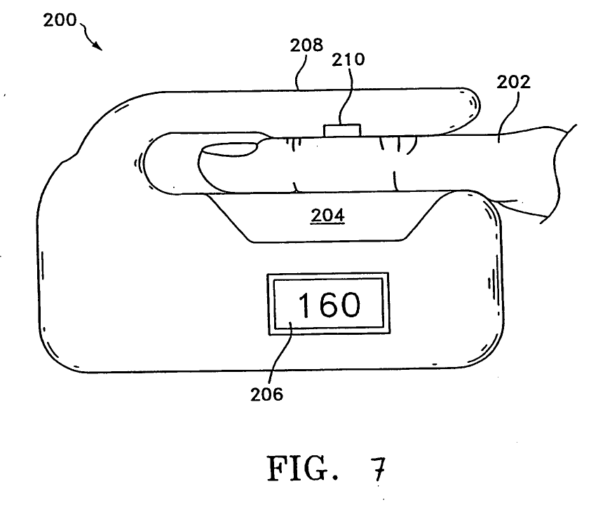

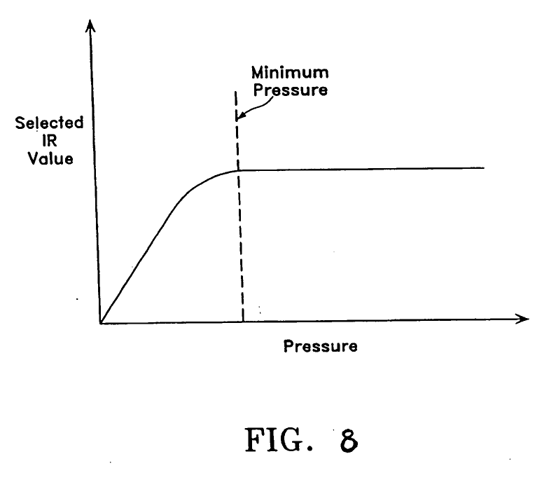

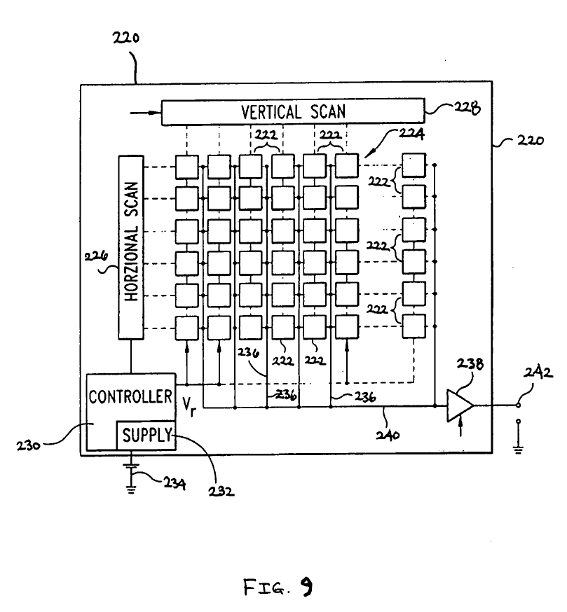

FIG. 7 shows a variation of this device 200 showing a finger of the user 202 over the ATR plate 204 with a display 206. Further shown in this variation 200 is a pressure maintaining component 208. Component 208 may be used to maintain a minimum threshold pressure on the body part which is to be used as the area to be measured. Generally, a variance in the pressure does not shift the position of the detected IR spectra, but it may affect the sensitivity of the overall device. The appropriate pressure will vary with, e.g., the size of the ATR plate and the like. A constant pressure above that minimum threshold value is most desired.

The variation shown in FIG. 7 uses a simple component arm 208 to maintain pressure of the finger 202 on ATR plate 204. Other variations within the scope of this invention may include clamps and the like.

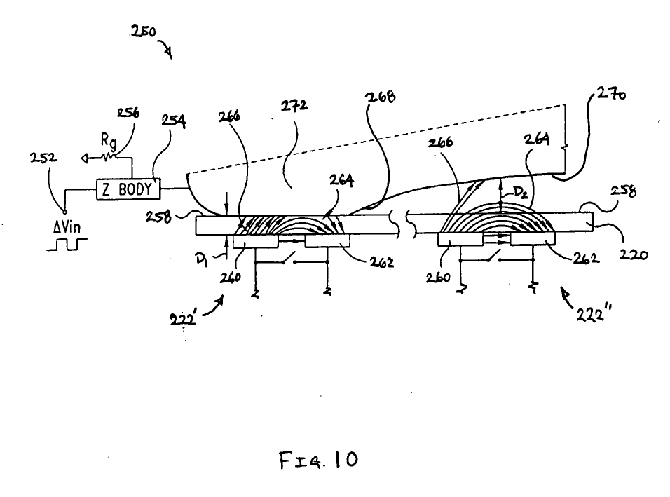

It should be apparent that once an appropriate pressure is determined for a specific design, the inventive device may include a pressure sensor integrated within ATR plate 204, as described above. Alternatively, a pressure sensor 210 may be integrated into the component arm 208 to measure adherence to that minimum pressure. It is envisioned that normally a pressure sensor such as 210 would provide an output signal which would provide a “no-go/go” type of signal to the user. Further, as shown in FIG. 8, the appropriate pressure may be achieved when using our device simply by increasing the pressure of the body part on the ATR crystal surface until a selected, measured IR value becomes constant.

Other analyte materials which have both referencing wavelengths and measuring wavelengths in the mid-IR range and that are found in the outer regions of the skin may also be measured using the inventive devices and procedures described herein. Respective signals may be compared using analog or digital computer devices. The signals are then used to calculate analyte values such as blood glucose concentration using various stored calibration values. The resulting calculated values may then be displayed. We also note that, depending upon the design of a specific variation of a device made according to the invention, periodic at least an initial calibration of the device, using typical blood sample glucose determinations, may be necessary or desirable.

In general, the inventive device described above may be used in the following manner: a skin surface on a human being, for instance, the skin of the finger, is placed on the ATR plate. The skin surface is radiated with an IR beam having components at least in the two IR regions as described above as the “referencing wavelength” and the “measuring wavelength.” The beam which ultimately is reflected out of the ATR plate then contains information indicative of the blood glucose level in the user. As noted above, it is also desirable to maintain that skin surface on the ATR plate at a relatively constant pressure that is typically above a selected minimum pressure. This may be done manually or by measuring and maintaining the pressure or monitoring the constancy of a selected IR value with the sensor assembly, as described above.

Additionally, as discussed above, the verification of a user's identification may be tied to the operation of the device such that a measurement will not be taken until the proper identification of the user has been verified by the device. Verification of a user's identification prior to use of the device may be incorporated as an optional safeguard. For instance, identification may be performed to ensure that the device, which is preferably calibrated to a specific user, may not be used inadvertently or otherwise by a person other than the specified user. Thus, the sensor assembly described above may be configured in one variation as a fingerprint sensing device.

A fingerprint sensing device may utilize the capacitance of the tissue in contact with the sensor assembly, as described in further detail in U.S. Pat. No. 6,512,381 (Kramer), which is incorporated herein by reference in its entirety. FIG. 9 shows an illustrative detailed top view of one variation of a sensor assembly which may be integrated into the ATR crystal, as described above. As shown, substrate 220 may have a plurality of sensor cells 222 located thereon in the form of an array 224. The array 224 may be organized in a column/row fashion to sense a fingerprint pattern of a finger, or a portion of a finger or some other body part, which may be placed onto the substrate 220. The array 224 may be driven by horizontal scan electronics 226 and also by vertical scan electronics 228 such that each individual sensor cell 222 can be selected on a known timing sequence under control of controller 230.

Each sensor cell 222 may be configured with electronics to detect the presence or absence of a ridge or a valley of a fingerprint pattern placed on top of the substrate 220. Controller 230 may be a separate processor integrated into the sensor assembly or alternatively it may be a processor 180 connected to light sensors 176, 178. Controller 230 may also include a power supply circuit 232 that receives power from a power source 234, which may also be the power source for operating the entire assembly of FIG. 6. Each sensing cell 222 may provide an output on electrical lines 236 of each individual column. The output received by each sensing cell 222 may be provided on the chip bus 240 which may be further connected to an output amplifier 238, which may then provide the composite output of sensor array 224 at terminal 242. The output of the sensor array 224 at terminal 242 may be provided to a fingerprint pattern recognition electronics which may then organize the signal into a fingerprint pattern and compare it to other fingerprint patterns to perform recognition of the fingerprint pattern which is placed on the substrate 230. The fingerprint pattern recognition may also be obtained by various methods and techniques as recognized by one of skill in the art.

In operation, the capacitive fingerprint sensing device through the sensor assembly may place a variable voltage on the user, e.g., the finger, of the individual whose fingerprint pattern is being sensed and verified. Applying a voltage onto the finger may serve to provide a variable charge transfer during sensing and also provides the variable capacitance to be sensed since placing a change in charge on a user's body may enhance the capacitive sensing capability, and thus the resulting measurement of the fingerprint. The application of the voltage increases the effectiveness and accentuates the measurable differences between a ridge and a valley when present near or over a sensor cell 222. The electrical connection to the user's body can be made by any number of methods; for instance, an electrical contact may be attached to the user's finger when placed upon the sensor assembly. Alternatively, a voltage may be placed upon the user's finger by a capacitive transfer. For instance, a large plate capacitor in substrate 230 may provide a plate to transfer the charge to the finger to alter the voltage while sensing the capacitive difference. In yet another alternative, a voltage change can be coupled to the user through another part of the body, e.g., an adjacent finger.

In one variation, the sensor circuit may include a negative feedback amplifier with the two plates of a feedback capacitor having a field between the two plates. The field between the two plates may be varied by the ridges and valleys defining the fingerprint of the user's finger. The sensed capacitance will be greater when a valley is over a sensor as opposed to having a ridge present over a sensor, which will detect a lower capacitance relative to the capacitance of a valley.

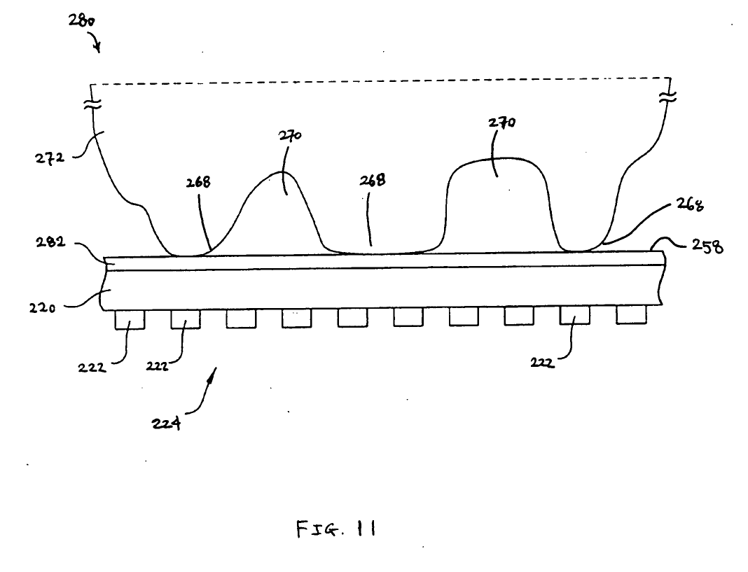

As seen in a detail schematic view of an example of sensor assembly 250 in FIG. 10, a voltage may be applied at a terminal 252. As illustrated, the user's body may have an impedance value represented as an overall impedance value Z BODY 254 between terminal 252 and the user's finger 272. The user's body may also have a resistance to ground, R_{g}, 256 which may vary between users. Resistance 256 also varies based on the position of connection to the user's body. In this example, two adjacent sensor cells 222′, 222″ may be seen in which a first sensor cell 222′ has a ridge 268 directly over the cell 222′ and a second sensor cell 222″ has a valley directly over the cell 222″. When ridge 268 is placed upon the contact surface 258 of substrate or dielectric 220, the distance between ridge 268 and first sensor cell 222′ may be defined as D₁. Here, the presence of the ridge 272 interferes with the fringe capacitive field lines 264 and thereby reduces the value of the capacitance 266 between plates 260 and 262.

On the other hand, when valley 270 of finger 272 is present over the adjacent second cell 222″, the finger 272 is separated by a distance D₂ from contact surface 258 of substrate 220. Depending upon the distance D₂, there will likely be little or no interference between the fringe capacitive field lines 264 extending between plates 260 and 262. In either case, the interference will be lower relative to the interference presented by a ridge 268 over a sensor cell. With valley 270 present and the finger 272 spaced a farther distance from the plates 260, 262, the input capacitance is relatively smaller when compared with the larger input capacitance when ridge 268 is present.

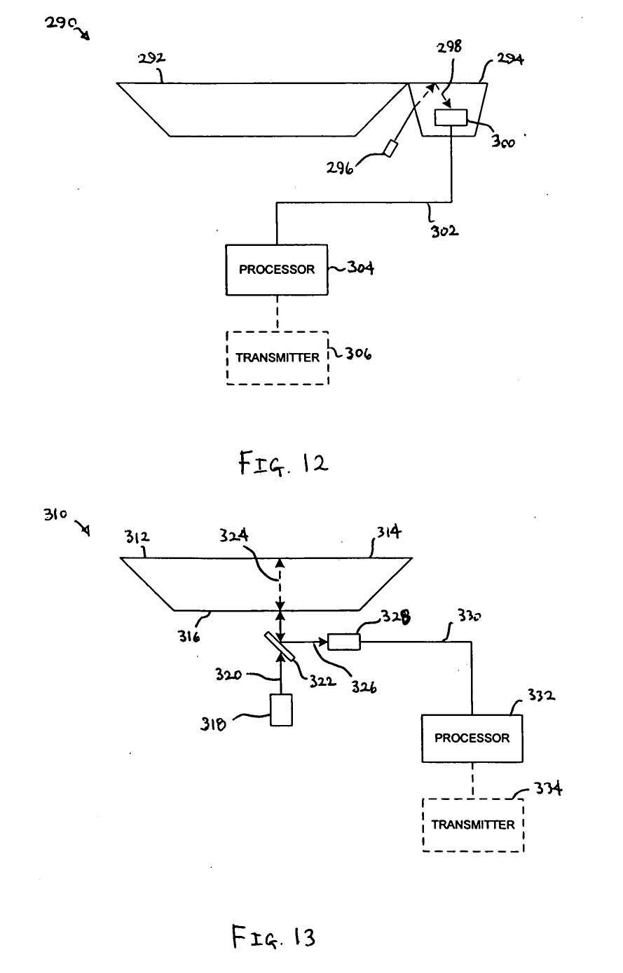

Thus, as shown in the assembly 280 of FIG. 11, when finger 272 is placed onto contact surface 258 of layer 282, a number of ridges 268 and valleys 270 defining the user's fingerprint is detected by the individual sensors 222 within sensor array 224. The capacitive values detected by sensor cells 222 will be a value unique to a particular user. A specific measurement device may therefore be calibrated to activate or measure glucose levels once the corresponding capacitive values of a specific user's fingerprint has been verified.

Aside from the use of capacitive sensing for user identification, another variation may utilize infrared light to illuminate the user's finger to capture a reflected fingerprint as an image. This captured image may be compared to a stored fingerprint image of a specified person to verify the user's identification. FIG. 12 shows a schematic illustration of infrared sensor assembly 290. ATR crystal 292 may be utilized for glucose measurement, as described above, while sensor assembly 294 may be used to detect the user's fingerprint. Any of the sensor assembly configurations described above or contemplated by one of ordinary skill in the art may be utilized for integrating the sensor assembly 294 with the ATR crystal 292. Light source 296, e.g., an LED, may be the same source used to transmit the light into ATR crystal 292 or it may be a separate light source. In either case, light source 296 preferably emits light in the visible or infrared (or mid-infrared) wavelength regions. The emitted light may reflect off a user's finger and this reflected light 298 may be incident upon a photosensor or light detector 300, e.g., a CCD or CMOS imaging system. The incident light may then be transmitted as electronic signals via electrical line 302 to a processor 304 for image processing and comparison. An image of a specified user's fingerprint may be stored in memory within processor 304 or from a separate integrated memory module for use in comparison by processor 304 with the detected fingerprint image. A detected image matched with the stored image may then allow for use of the detection and measurement functions (e.g., glucose detection and measurement) of the device. Optionally, a transmitter 306 may be included with IR sensor assembly 290 for transmitting a detected fingerprint image to an external receiving unit where the detected image may be compared to an externally stored image, the results of which may then be transmitted back to the IR assembly 290 for processing by processor 304.

An alternative variation may be seen in the schematic illustration in FIG. 13. In IR sensor assembly 310, light source 318 may transmit an emitted light 320 directly into ATR crystal 312. As the light passes through lower ATR surface 316, it may reflect off the person's finger resting upon ATR upper surface 314 and this reflected light 324 may then pass again through lower surface 316. Reflected light 324 may be directed, e.g., via beam splitter 322, as light 326 to light sensor 328, which may be any of the variations described above. The detected received light 326 may then be transmitted via electrical line 330 to processor 332. As above, the processed fingerprint image may then be transmitted via an optional transmitter 334, as above.

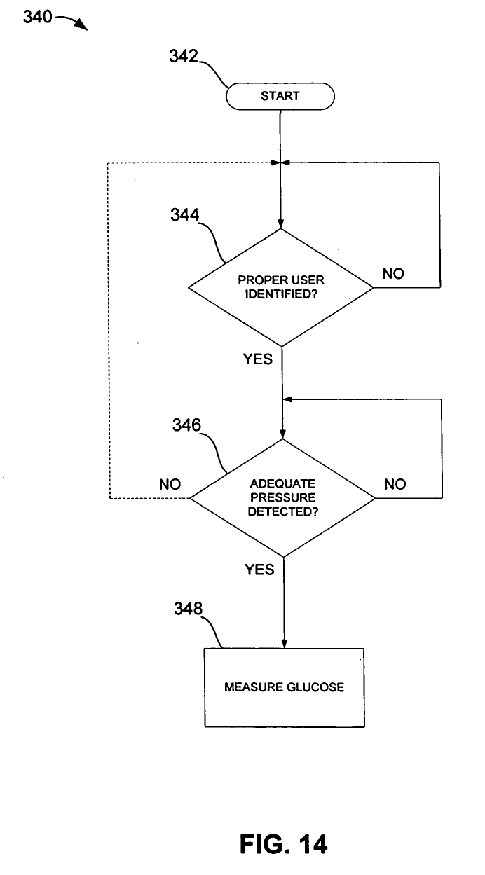

As mentioned above, both user identification and pressure may be detected by appropriate sensors integrated into the glucose measurement device. FIG. 14 shows one variation of a detection algorithm 340 which may be utilized with the device sensors. Step 342 indicates the start of the process. The user may place a finger onto the ATR crystal and/or sensor assembly. The user's identification may then be detected or sensed, as shown in step 344. If a positive match is not detected, i.e., an improper fingerprint measurement has occurred or an unauthorized user has attempted to use the device, the device may be configured to not operate until a positive match has been detected, as shown. If the match is positive, i.e., an authorized user having the appropriate stored profile is detected, the device may then detect whether the user is exerting the adequate amount of pressure onto the ATR crystal, as indicated by step 346. If the user is not exerting the appropriate amount of pressure, the device may be configured to not measure or activate until the minimum adequate pressure is sensed, as shown. Alternatively, the device may be configured to reset and re-verify the user's identification first before detecting the pressure again, as shown. This optional step may be utilized to prevent an authorized user from activating the device with his/her verified identification and then passing the device to an unauthorized user for glucose or analyte measurement. Finally, once the adequate pressure has been detected, the device may operate as described above to detect and measure the analyte or glucose of the user, as indicated by step 348.

The variation shown in FIG. 14 is intended to be illustrative and is not intended to be limiting. Accordingly, various combinations as well as variations on the order of detection and/or identification may be utilized, depending upon the desired results.

This invention has been described and specific examples of the invention have been portrayed. The use of those specifics is not intended to limit the invention in any way. Additionally, to the extent there are variations of the invention with are within the spirit of the disclosure and yet are equivalent to the inventions found in the claims, it is our intent that this patent will cover those variations as well.

1. An analyte level measurement device having at least one integrated sensor, comprising:
an infrared source for emitting an IR beam into an ATR plate, the IR beam having components at least in the region of a referencing wavelength and a measuring wavelength, the ATR plate having a measurement surface for contact with a skin surface of a user and for directing the IR beam against the skin surface, at least one IR sensor for measuring an absorbance of at least the referencing wavelength and the measuring wavelength; and
a sensor assembly having a contact surface, the sensor assembly being configured to detect at least one biometric feature indicative of the user.

2. The device of claim 1 wherein the ATR plate is configured to permit multiple internal reflections against the measurement surface prior to measuring the absorbance.

3. The device of claim 2 wherein the ATR plate is configured for about 3 about 25 internal reflections against the measurement surface.

4. The device of claim 1 further comprising a pressure measurement sensor situated to measure a pressure of the skin surface against the ATR plate.

5. The device of claim 1 wherein the analyte is glucose and the referencing wavelength is between about 8.25 micrometers and about 8.75 micrometers.

6. The device of claim 1 wherein the analyte is glucose and the measuring wavelength is between about 9.50 micrometers and about 10.00 micrometers.

7. The device of claim 1 further comprising a processor for comparing the measuring wavelength to the referencing wavelength.

8. The device of claim 1 further comprising a display for displaying a measurement selected from the group consisting of an analyte concentration, an analyte amount, and a trace presence of an analyte.

9. The device of claim 1 wherein the infrared source is an LED.

10. The device of claim 1 wherein the infrared source is a non-laser source.

11. The device of claim 1 wherein the contact surface is flush with the measurement surface.

12. The device of claim 1 wherein the sensor assembly is adjacent to a single side of the ATR plate.

13. The device of claim 1 wherein the sensor assembly is adjacent to at least two sides of the ATR plate.

14. The device of claim 1 wherein the sensor assembly is surrounded by the ATR plate.

15. The device of claim 1 wherein the sensor assembly comprises a plurality of sensor cells arranged such that at least one sensor cell is adapted to detect a capacitive effect from at least a portion of the skin surface.

16. The device of claim 15 wherein the plurality of sensor cells is arranged in an array.

17. The device of claim 15 wherein each sensor cell is adapted to detect the presence of a ridge or a valley from the skin surface.

18. The device of claim 1 wherein the sensor assembly is adapted to detect an image of the biometric feature.

19. The device of claim 18 wherein the infrared source is adapted to illuminate the biometric feature for measurement of the feature.

20. The device of claim 19 further comprising a photosensor for detecting the image.

21. The device of claim 20 wherein the photosensor is a solid state imager.

22. The device of claim 18 wherein the sensor assembly is further adapted to compare the detected image against a stored image.

23. The device of claim 18 further comprising a transmitter for transmitting the image to an external receiving device.

24. The device of claim 1 wherein the biometric feature is at least a portion of a fingerprint of the user.

25. A method for selectively determining an analyte level from a skin surface, comprising
contacting the skin surface against a measurement surface of an ATR plate;
measuring at least one biometric feature from the skin surface;
comparing the measured biometric feature against a predetermined biometric feature indicative of a predetermined user;
if the measured biometric feature matches the predetermined biometric feature, then irradiating the skin surface with an IR beam having components at least in a region of a referencing wavelength and a measuring wavelength through the ATR plate to produce a reflected IR beam indicative of the analyte level; and
detecting and quantifying the referencing wavelength and the measuring wavelength components in the reflected IR beam.

26. The method of claim 25 further comprising detecting a pressure exerted by the skin surface against the ATR plate prior to detecting and quantifying.

27. The method of claim 25 wherein contacting the skin surface against the measurement surface further comprises contacting the skin surface against a contact surface of a sensor assembly.

28. The method of claim 25 wherein measuring at least one biometric feature comprises measuring a capacitance of at least a portion of the skin surface, wherein the measured capacitance is unique to at least a portion of the skin surface.

29. The method of claim 28 wherein comparing the measured biometric feature comprises comparing the measured capacitance against a predetermined capacitance indicative of the predetermined user.

30. The method of claim 25 wherein measuring at least one biometric feature comprises illuminating at least a portion of the skin surface and detecting a reflected image.

31. The method of claim 30 wherein comparing the measured biometric feature comprises comparing the reflected image against a predetermined image indicative of the predetermined user.

32. The method of claim 31 wherein the reflected image is transmitted to an external receiving unit for comparison against the predetermined image.

33. The method of claim 25 further comprising maintaining the skin surface against the ATR plate at an adequate pressure while irradiating the skin surface.

34. The method of claim 25 further comprising maintaining the skin surface against the ATR plate at a constant and above a selected minimum pressure while irradiating the skin surface.

35. The method of claim 25 further comprising normalizing the referencing wavelength and the measuring wavelength components prior to contacting the skin surface.

36. The method of claim 25 wherein the referencing wavelength is between about 8.25 micrometers and about 8.75 micrometers.

37. The method of claim 25 wherein the measuring wavelength is between about 9.50 micrometers and about 10.00 micrometers.
